(19) United States
(12) Patent Application Publication
(10) Pub. No.: US 2010/0210852 A1
(43) Pub. Date: Aug. 19, 2010

(54) PROCESS FOR THE PREPARATION OF CANDESARTAN CILEXETIL

(75) Inventors: Keshav Deo, Gujarat (IN); Sanjay Desai, Gujarat (IN); Dhiraj Mohansinh Rathod, Gujarat (IN); Lalitkumar Keshavial Katariya, Gujarat (IN); Nilesh Vashrambhai Bhimani, Gujarat (IN)

Correspondence Address: MERCHANT & GOULD PC, P.O. BOX 2903, MINNEAPOLIS, MN, 55402-0903, US

(73) Assignee: Alembic Limited, Vadodara (IN)

(21) Appl. No.: 12/668,618

(22) PCT Filed: Aug. 30, 2007
(86) PCT No.: PCT/IN2007/000378

§ 371 Date: Jan. 11, 2010

(30) Foreign Application Priority Data
Jul. 11, 2007 (IN) 1331/MUM/2007

(51) Int. Cl. C07D 257/04

(52) U.S. Cl. 548/253

(57) ABSTRACT

The present invention relates to an improved process for the preparation of tritylated candesartan acid of formula (I)

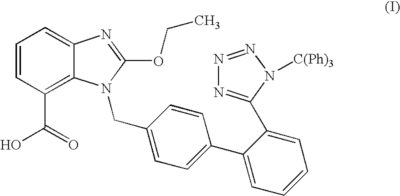

comprising a step of, reacting candesartan acid of formula (II)

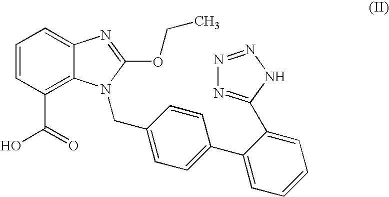

with trityl chloride in the presence of a base in a ketonic solvent.

### FIELD OF INVENTION

The present invention relates to an improved process for the preparation of Candesartan cilexetil. Particularly, the present invention relates to an improved process for the preparation of tritylated Candesartan acid of formula (I).

### BACKGROUND OF THE INVENTION

The chemical name of Candesartan Cilexetil is 1-[[(Cyclohexyloxy)carbonyl]oxy]ethyl 2-ethoxy-1-[[2-(1H-tetazole-5-yl)[1,1′-biphenyl-4-yl]methyl]-1H-benzimidazole-7-carboxylate. Its molecular formula is C₃₃H₃₄N₆O₆ and mol wt is 610.66. Candesartan Cilexetil is represented by structural formula (III)

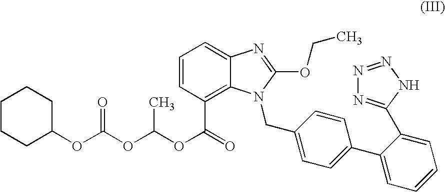

Candesartan Cilexteil is an ester prodrug of 2-ethoxy-1-[[2-(1H-tetrazole-5-yl)[1,1′-biphenyl-4-yl]methyl]-1H benzimidazole-7-carboxylic acid (candesartan), known as a potent Angiotensin II receptor antagonist. It is useful in the treatment of cardiovascular complaints such as hypertension and heart failure. Candesartan cilexetil is a white to off-white powder and is sparingly soluble in water and in methanol. It is marketed by AstraZeneca under tradename ATACAND®.

U.S. Pat. No. 5,196,444 describes a process of preparation of tritylated candesartan acid of formula (I) by reacting candesartan acid of formula (II) with trityl chloride in the presence of base in a solvent which is selected from halogenated hydrocarbons such as chloroform, methylene chloride and ethylene chloride, ethers such as dioxane and tetrahydrofuran, acetonitrile, pyridine to obtain tritylated candesartan acid of formula (I) in 66% yield after column chromatography. The yield obtained by this process is very low due to the presence of 10-20% impurities. Moreover, the purification of final product by chromatography is commercially not suitable and is cumbersome at an industrial scale.

U.S. Pat. No. 5,196,444 describes a process of preparation of Candesartan cilexetil in which it is formed by reacting 2-ethoxy-1-[[2′-(N-triphenylmethyltetrazol-5-yl)biphenyl-4-yl]methyl]benzimidazole-7-carboxylic acid in dimethylformamide with cyclohexyl-1-iodoethyl carbonate to form cilexetil trityl candesartan and its subsequent deprotection with a methanolic hydrochloric acid gives candesartan cilexetil in 47% yield after column chromatography. The yield obtained by this process is very low. Moreover, the purification of final product by chromatography is commercially not suitable and is cumbersome at an industrial scale.

U.S. Pat. No. 5,578,733, describes a process of preparation of candesartan cilexetil comprising deprotection of cilexetil trityl candesartan with mineral acids is done under substantially anhydrous conditions in the presence of alcohol. The purification of candesartan cilexetil involves a variety of extraction steps with solvents such as ethyl acetate, ethanol, and acetone prior to crystallizing candesartan cilexetil from aliphatic hydrocarbon such as hexane. Such purification process is tedious, laborious to perform and time consuming.

The complexity and high cost of the prior art procedures has created a need for an improved process for the preparation of tritylated candesartan acid of formula (I) and candesartan cilexetil. The present invention provides a solution to the problem presented by the prior art.

Through experimentation, the present inventors have observed that the tritylation step in the process for the preparation of tritylated Candesartan acid is sensitive and directly related to the formation of impurities, quality and yield of the final product. Therefore, we directed our research work toward developing a process which avoids these difficulties during tritylation step for the preparation of tritylated candesartan acid of formula (I).

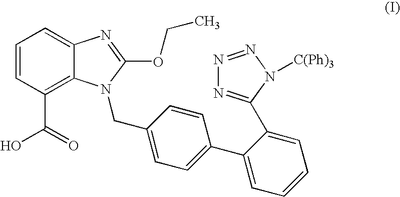

Surprisingly, the present inventors have found that the use of ketonic solvent during tritylation step provides substantial increase in yield and quality of tritylated candesartan acid of formula (I). Further, the process does not involve additional step of purification of tritylated candesartan acid of formula (I).

### OBJECT OF THE INVENTION

A primary object of the present invention is to provide an improved process for the preparation of tritylated candesartan acid of formula (I).

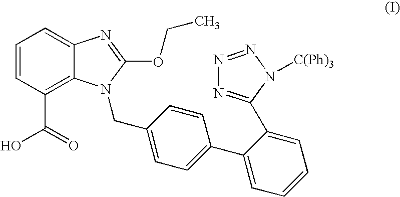

Another object of the present invention is to provide a process for the preparation of Candesartan Cilexetil.

Further another object of the present invention is to provide an improved process for preparation of tritylated candesartan acid of formula (I), which is simple, easy to handle and feasible at commercial scale.

Yet another object of the present invention is to provide an improved process for the preparation of tritylated candesartan acid of formula (I)

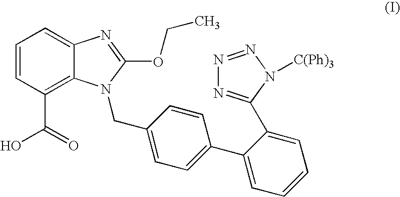

comprising a step of, reacting candesartan acid of formula (II)

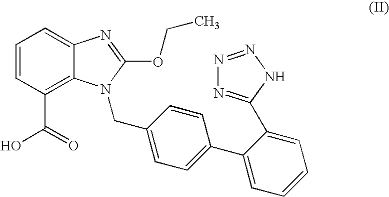

with trityl chloride in the presence of a base in a ketonic solvent.

Yet another object of the present invention is to provide an improved process for the preparation of candesartan cilexetil of formula (III),

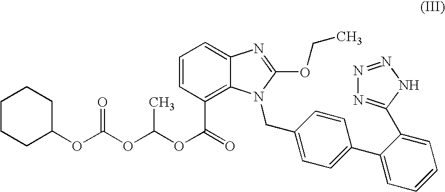

comprising steps of,
a) reacting candesartan acid of formula (II)

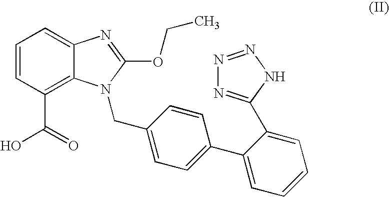

with trityl chloride in the presence of a base in a ketonic solvent to obtain tritylated candesartan acid of formula (I)
b) reacting tritylated candesartan acid of formula (I)

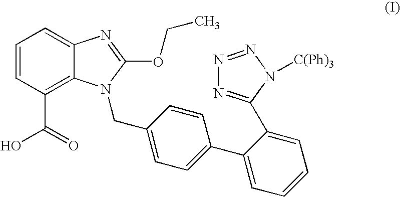

with cyclohexyl 1-chloroethylcarbonate in the presence of a base, catalyst in a solvent to obtain tritylated candesartan cilexetil of formula (IV)
c) deprotecting tritylated candesartan cilexetil of formula (IV)

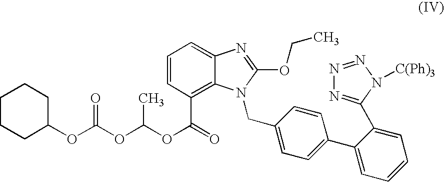

with inorganic acid in the presence of alcohol to obtain candesartan cilexetil

Another object of the present invention is to provide an improved process for preparation of Candesartan Cilexetil, which is simple, easy to handle and feasible at commercial scale.

### SUMMARY OF THE INVENTION

The present invention provides an improved process for the preparation of tritylated candesartan acid of formula (I)

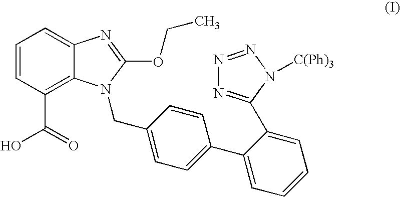

comprising a step of, reacting candesartan acid of formula (II)

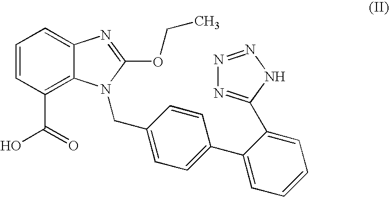

with trityl chloride in the presence of a base in a ketonic solvent.

Another aspect of the present invention is to provide an improved process for the preparation of candesartan cilexetil of formula (III),

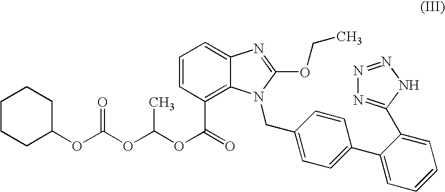

comprising steps of,
a) reacting candesartan acid of formula (II)

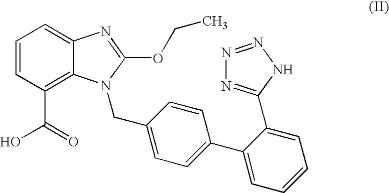

with trityl chloride in the presence of a base in a ketonic solvent to obtain tritylated candesartan acid of formula (I)
b) reacting tritylated candesartan acid of formula (I)

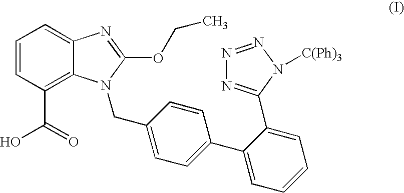

with cyclohexyl 1-chloroethylcarbonate in the presence of base, catalyst in a solvent to obtain tritylated candesartan cilexetil of formula (IV)
c) deprotecting tritylated candesartan cilexetil of formula (IV)

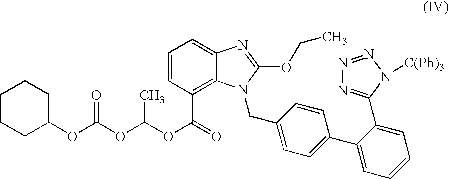

with inorganic acid in the presence of alcohol to obtain candesartan cilexetil

### DETAILED DESCRIPTION OF THE INVENTION

In accordance with the object of the present invention one embodiment provides an improved process for the preparation of tritylated candesartan acid of formula (I)

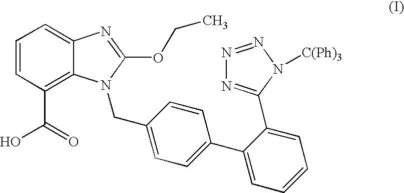

comprising a step of, reacting candesartan acid of formula (II)

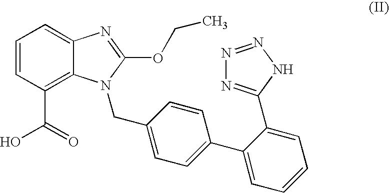

with trityl chloride in the presence of a base in a ketonic solvent.

The suitable base is selected from inorganic base and organic base. The example of an inorganic base are potassium carbonate, calcium carbonate, sodium carbonate, sodium hydroxide, sodium hydrogen carbonate, sodium amide, sodium hydride and the like or mixture thereof. The example of an organic base are triethylamine, tripropylamine, pyridine, quinoline and the like or mixture thereof.

The ketonic solvent as mentioned hereinabove is selected from a group comprising of acetone, methyl isobutyl ketone (MIBK), methyl ethyl ketone (MEK) and the like or mixture thereof. The preferred solvent is acetone.

The reaction can be carried out at reflux temperature. After completion of the reaction, reaction mixture is cooled at ambient temperature followed by addition of D. M. water and stir for one hour. The reaction mixture is filtered and washed with mixture of acetone and D. M. water. The solid was dried to obtain tritylated Candesartan acid of formula (I).

Another embodiment of the present invention provides an improved process for the preparation of candesartan cilexetil of formula (III),

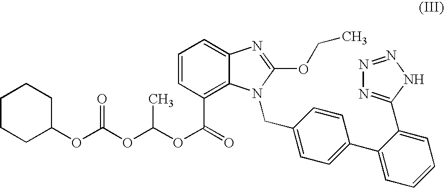

comprising steps of,
a) reacting candesartan acid of formula (II)

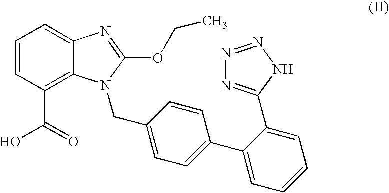

with trityl chloride in the presence of a base in a ketonic solvent to obtain tritylated candesartan acid of formula (I)
b) reacting tritylated candesartan acid of formula (I)

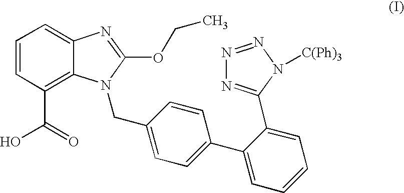

with cyclohexyl 1-chloroethylcarbonate in the presence of base, catalyst in a solvent to obtain tritylated candesartan cilexetil of formula (IV)
c) deprotecting tritylated candesartan cilexetil of formula (IV)

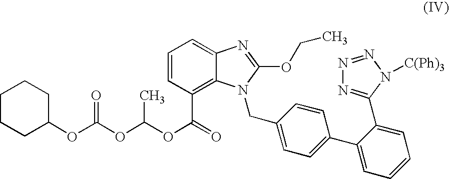

with inorganic acid in the presence of alcohol to obtain candesartan cilexetil as shown in the synthetic representation given below in Scheme-I.

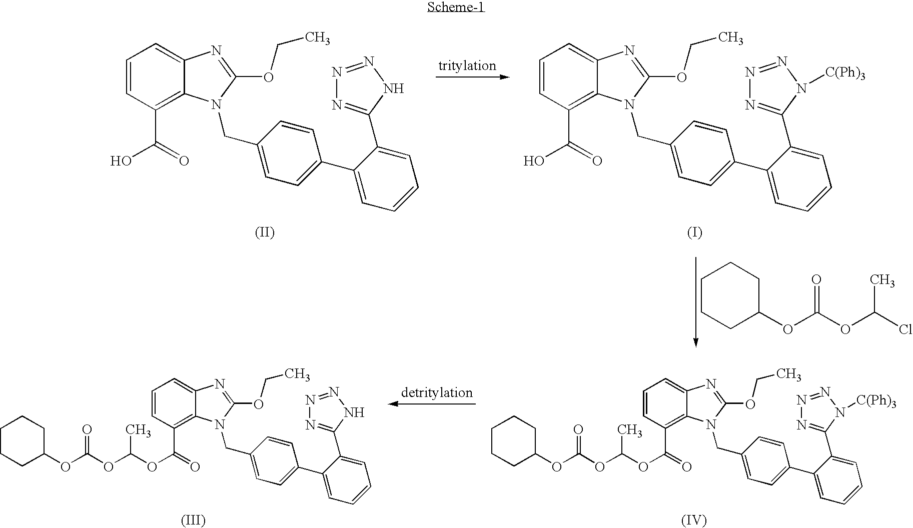

The suitable base in step (a) is selected from inorganic base and organic base. The example of an inorganic base are potassium carbonate, calcium carbonate, sodium carbonate, sodium hydroxide, sodium hydrogen carbonate, sodium amide, sodium hydride and the like or mixture thereof. The example of an organic base are triethylamine, tripropylamine, pyridine, quinoline and the like or mixture thereof.

The ketonic solvent as mentioned hereinabove is selected from a group comprising of acetone, methyl isobutyl ketone (MIBK), methyl ethyl ketone (MEK) and the like or mixture thereof. The preferred solvent is acetone.

The reaction in step (a) can be carried out at reflux temperature. After compilation of the reaction, reaction mixture is cooled at ambient temperature followed by addition of D. M. water and stir for one hour. The reaction mixture is filtered and washed with mixture of acetone and D. M. water. The solid was dried to obtain tritylated Candesartan acid of formula (I).

The suitable base mentioned hereinabove in step (b) include but not limited to an inorganic base such as potassium carbonate, calcium carbonate, sodium carbonate, sodium hydroxide, sodium hydrogen carbonate, sodium amide, sodium hydride and the like or mixture thereof; and an organic base such as triethylamine, tripropylamine, pyridine, quinoline and the like or mixture thereof.

The suitable solvent mentioned hereinabove in step (b) include but not limited to ethers such as dioxane, tetrahydrofuran, ethylene glycol dimethyl ether and the like or mixture thereof; aromatic hydrocarbons such as toluene, xylene and the like or mixture thereof; lower alcohols such as methanol, ethanol, isopropanol and the like or mixture thereof; polar solvents such as dimethylformamide (DMF), dimethyl sulfoxide (DMSO), acetonitrile, dimethylacetamide and the like or mixture thereof.

The suitable reaction accelerator or catalyst mentioned hereinabove in step (b) include but not limited to an alkali metal iodide such as potassium iodide, sodium iodide.

The reaction in step (b) can be carried out at 60-70° C. After completion of the reaction, reaction mixture was cooled at ambient temperature. The reaction mixture was poured in water at 0-10° C. and stirred for one hour. The mixture was filtered and washed with D. M. water. A mixture of wet cake and acetone was stirred and heated for 30 minutes at 55-60° C. The reaction mixture was cooled and stirred at ambient temperature for 30 minutes. The mixture was filtered and washed with acetone. The solid was dried to obtain tritylated Candesartan cilexetil of formula (IV).

The suitable inorganic acid mentioned hereinabove in step (c) include but not limited to an inorganic acid such as hydrochloride, sulphuric acid, nitric acid.

The suitable solvent mentioned hereinabove in step (c) include but not limited to alcohol such as methanol, ethanol, isopropanol and the like or mixture thereof.

After the completion of the reaction, sodium bicarbonate solution was added to the reaction mixture and organic layer was separated. Aqueous layer was extracted with methylene dichloride (MDC). Both organic layers were combined and washed brine solution. MDC was distilled out under vacuum to give residue. A mixture of rectified spirit and cyclohexane was added to the residue and stirred for 3 hours. The mixture was filtered and washed with mixture of rectified spirit and cyclohexane. The solid was dried to obtain Candesartan cilexetil.

The purification of crude candesartan cilexetil is carried out in the mixture of acetone and water to obtain pure candesartan cilexetil.

The present inventors have specifically observed distinct advantages of ketonic solvents in terms of yield and purity. When acetone is used as solvent it provides the tritylated candesartan acid with substantial increase in yield and purity. The comparison between prior art solvent and present invention solvent:

| S. No | Solvent | Yield (%) | Purity (%) |
|---|---|---|---|
| 1 | MDC (Prior art solvent) | 60-65 | 80-85 |
| 2 | Acetone (Ketonic solvent) | 88-90 | 98-99 |

Further, the present invention has following advantages over prior art:

(i) It provides a process which is operationally simple and industrially applicable.
(ii) This process avoids the use of dry HCl gas which is a tedious process.
(iii) It involves less reaction time then prior art process.
(iv) It controls the formation of impurities in tritylation step.
(v) It controls the formation of impurities in detritylation step.

The process of the present invention is described by the following examples, which are illustrative only and should not be construed so as to limit the scope of the invention in any manner.

### Examples-1

### Preparation of Tritylated Candesartan Acid (Acetone)

A mixture of Candesartan acid, triethylamine and acetone was heated to reflux temperature at 55-60° C. To this trityl chloride solution in acetone was added and refluxed it for 4-8 hours. The reaction mixture was cooled at ambient temperature followed by addition of D. M. water and stirred for one hour. The reaction mixture was filtered and washed with mixture of acetone and D. M. water. To the solid, D. M water was added and stirred for 30 minutes at ambient temperature. The mixture was filtered and washed with D. M. water. The solid was dried to obtain tritylated Candesartan acid.

Yield: 90%

Purity: 99%

### Examples-2

### Preparation of Tritylated Candesartan Acid (MIBK)

A mixture of Candesartan acid, triethylamine and methyl isobutyl ketone (MIBK) was heated to reflux temperature at 55-60° C. To this trityl chloride solution in MIBK was added and refluxed it for 4-8 hours. The reaction mixture was cooled at ambient temperature followed by addition of D. M. water and stirred for one hour. The reaction mixture was filtered and washed with mixture of acetone and D. M. water. To the solid, D. M water was added and stirred for 30 minutes at ambient temperature. The mixture was filtered and washed with D. M. water. The solid was dried to obtain tritylated Candesartan acid.

Yield: 89%

Purity: 98.5%

### Examples-3

### Preparation of Tritylated Candesartan Acid (MEK)

A mixture of Candesartan acid, triethylamine and methyl ethyl ketone (MEK) was heated to reflux temperature at 55-60° C. To this trityl chloride solution in MEK was added and refluxed it for 4-8 hours. The reaction mixture was cooled at ambient temperature followed by addition of D. M. water and stirred for one hour. The reaction mixture was filtered and washed with mixture of acetone and D. M. water. To the solid, D. M water was added and stirred for 30 minutes at ambient temperature. The mixture was filtered and washed with D. M. water. The solid was dried to obtain tritylated Candesartan acid.

Yield: 88%

Purity: 98%

### Examples-4

### Preparation of Tritylated Candesartan Cilexetil

A mixture of trityl Candesartan, dimethylformamide (DMF) and potassium carbonate at was heated at 60-70° C. Cyclohexyl 1-chloroethylcarbonate was added at 55-60° C. to the reaction mixture and maintain for 3 hours at 55-60° C. The reaction mixture was cooled at ambient temperature. The reaction mixture was poured in water at 0-10° C. and stirred for one hour at 0-10° C. The mixture was filtered and washed with D. M. water. A mixture of wet cake and acetone was stirred and heated for 30 minutes at 55-60° C. The reaction mixture was cooled and stirred at ambient temperature for 30 minutes. The mixture was filtered and washed with acetone. The solid was dried to obtain tritylated Candesartan cilexetil.

Yield: 92-95%

### Examples-5

### Preparation of Candesartan Cilexetil

A mixture of cilexetil trityl Candesartan in MDC was cooled at −10 to −5° C. A mixture of, methanol and hydrochloric acid was added to the reaction mixture at −10 to −5° C. and maintained for 3 hours. Sodium bicarbonate solution was added to the reaction mixture and organic layer was separated. Aqueous layer was extracted with MDC. Both organic layers were combined and washed brine solution. MDC was distilled out under vacuum to give residue. A mixture of rectified spirit and cyclohexane was added to the residue and stirred for 3 hours. The mixture was filtered and washed with mixture of rectified spirit and cyclohexane. The solid was dried to obtain Candesartan cilexetil.

### Purification of Crude Candesartan Cilexetil

A mixture of crude candesartan cilexetil, acetone and water was stirred at 55-60° C. The hot solution was filtered and filtrate was cooled at ambient temperature for 3 hours. The mixture was filtered and washed with mixture of acetone and water. The solid was dried to obtain pure Candesartan cilexetil.

Yield: 68-72%

1. A process for the preparation of tritylated candesartan acid of formula (I) 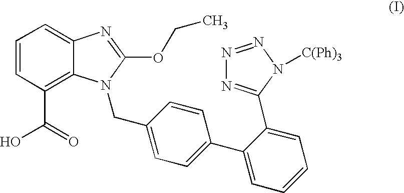
comprising a step of, reacting candesartan acid of formula (II) 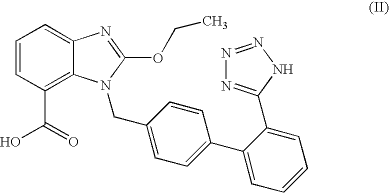
with trityl chloride in the presence of a base in a ketonic solvent.

2. A process as claimed in claim 1, wherein said base is selected from a group comprising of inorganic base and organic base.

3. A process as claimed in claim 2, wherein said inorganic base is selected from a group comprising of potassium carbonate, calcium carbonate, sodium carbonate, sodium hydroxide, sodium hydrogen carbonate, sodium amide and sodium hydride or mixture thereof.

4. A process as claimed in claim 2, wherein said organic base is selected from a group comprising of triethylamine, tripropylamine, pyridine and quinoline or mixture thereof.

5. A process as claimed in claim 1, wherein said ketonic solvent is selected from a group comprising of acetone, methyl isobutyl ketone (MIBK) and methyl ethyl ketone (MEK) or mixture thereof.

6. A process for the preparation of candesartan cilexetil of formula (III), 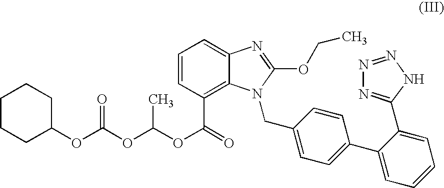
comprising steps of,
a) reacting candesartan acid of formula (II) 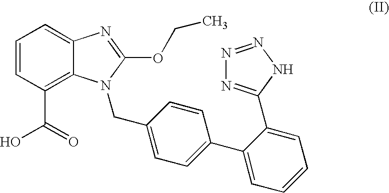
with trityl chloride in the presence of a base in a ketonic solvent to obtain tritylated candesartan acid of formula (I)
b) reacting tritylated candesartan acid of formula (I) 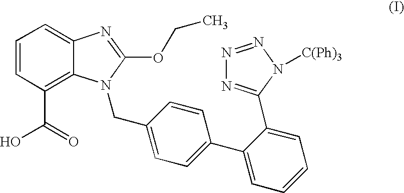
with cyclohexyl 1-chloroethylcarbonate in the presence of a base, catalyst in a solvent to obtain tritylated candesartan cilexetil of formula (IV)
c) deprotecting tritylated candesartan cilexetil of formula (IV) 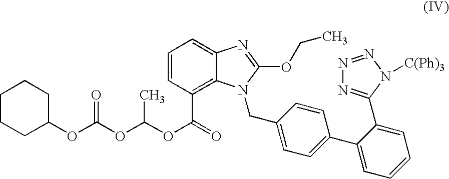
with inorganic acid in the presence of alcohol to obtain candesartan cilexetil.

7. A process as claimed in claim 6, wherein said base in step (a) is selected from a group comprising of inorganic base and organic base.

8. A process as claimed in claim 7, wherein said inorganic base is selected from a group comprising of potassium carbonate, calcium carbonate, sodium carbonate, sodium hydroxide, sodium hydrogen carbonate, sodium amide and sodium hydride or mixture thereof.

9. A process as claimed in claim 7, wherein said organic base is selected from a group comprising of triethylamine, tripropylamine, pyridine and quinoline or mixture thereof.

10. A process as claimed in claim 6, wherein said ketonic solvent in step (a) is selected from a group comprising of acetone, methyl isobutyl ketone (MIBK) and methyl ethyl ketone (MEK) or mixture thereof.

11. A process as claimed in claim 6, wherein said base in step (b) is selected from inorganic base and organic base.

12. A process as claimed in claim 11, wherein said inorganic base is selected from a group comprising of potassium carbonate, calcium carbonate, sodium carbonate, sodium hydroxide, sodium hydrogen carbonate, sodium amide and sodium hydride or mixture thereof.

13. A process as claimed in claim 11, wherein said organic base is selected from a group comprising of triethylamine, tripropylamine, pyridine and quinoline or mixture thereof.

14. A process as claimed in claim 6, wherein said solvent in step (b) is selected from a group comprising of dioxane, tetrahydrofuran, ethylene glycol dimethyl ether, toluene, xylene, methanol, ethanol, isopropanol, dimethylformamide (DMF), dimethyl sulfoxide (DMSO), acetonitrile and dimethylacetamide or mixture thereof.

15. A process as claimed in claim 6, wherein said catalyst in step (b) is selected from a group comprising of an alkali metal iodide.

16. A process as claimed in claim 15, wherein said alkali metal iodide is selected from a group comprising of potassium iodide, sodium iodide.

17. A process as claimed in claim 6, wherein said reaction in step (b) is carried out at temperature 60-70° C.

18. A process as claimed in claim 6, wherein said inorganic solvent in step (c) is selected from a group comprising of hydrochloride, sulphuric acid and nitric acid.

19. A process as claimed in claim 6, wherein said alcohol in step (c) is selected from a group comprising of methanol, ethanol and isopropanol or mixture thereof.
